(19) United States
(12) Patent Application Publication
(10) Pub. No.: US 2022/0370368 A1
(43) Pub. Date: Nov. 24, 2022

(54) ORAL PHARMACEUTICAL COMPOSITION COMPRISING A MELT-AGGLOMERATED ACTIVE INGREDIENT CORE

(71) Applicant: Hermes Pharma GmbH, Pullach (DE)

(72) Inventors: Axel WILDZEISZ, Pullach (DE); Josef HAALA, Pullach (DE); Wolfgang SCHIEMENZ, Pullach (DE)

(21) Appl. No.: 17/755,495

(22) PCT Filed: Oct. 29, 2020
(86) PCT No.: PCT/EP2020/080447

§ 371 Date: Apr. 29, 2022

(30) Foreign Application Priority Data
Oct. 31, 2019 (EP) 19206596.9

(51) Int. Cl. A61K 9/50; A61K 9/16; A61K 31/138; A61K 31/5386; A61K 31/155; A61K 31/522; A61K 31/167; A61K 31/192; A61K 31/549; A61K 9/48

(52) U.S. Cl. CPC A61K 9/5015; A61K 9/1694; A61K 31/138; A61K 31/5386; A61K 31/155; A61K 31/522; A61K 31/167; A61K 31/192; A61K 31/549; A61K 9/485; A61K 9/4858

(57) ABSTRACT

The invention relates to coated particles with a taste-masked drug substance. The particles comprise a core with a melt-agglomerated active pharmaceutical ingredient (API), optionally a thermolabile API, and a coating comprising a triglyceride and a surfactant. The particles exhibit immediate drug release and a storage-stable release profile. Moreover, the invention provides a hot-melt granulation and hot-melt coating method for manufacturing such coated particles, and pharmaceutical compositions comprising the coated particles. The method allows the granulation of APIs with small particle sizes (e.g., mean particle size below 150 μm, or even below 100 μm or 50 μm) into core particles, as well as coating said core particles, at moderate temperatures, thereby preventing the degradation of thermolabile active pharmaceutical ingredients.

### FIELD OF THE INVENTION

The present invention relates to the field of pharmaceutics, and concerns oral drug formulations, their manufacture and their use.

### BACKGROUND OF THE INVENTION

Most patients prefer orally administered medicaments over other routes of administration. However, in order to be acceptable to patients, an oral drug product must be easily swallowed and without unpleasant or bitter taste or other undesirable organoleptic properties. Unfortunately, many active ingredients, and in particular many drug substances, also commonly referred to as active pharmaceutical ingredients (APIs), exhibit a rather poor taste, said taste perception often being aggravated by APIs with small particle sizes and/or high water-solubility. A poor taste often involves a significant level of bitterness but may also entail other unpleasant sensations such as a burning stinging, metallic, or astringent mouthfeel.

For instance, Dimenhydrinate (DMH) is an antiemetic agent used in many countries in the management of symptoms associated with nausea, vomiting and/or dizziness, e.g. as a result of motion sickness. As an antiemetic agent, dimenhydrinate is typically administered at a dose of 20 mg or 40 mg about one to four times daily. Dimenhydrinate is a combination, or a salt of two drugs (diphenhydramine and 8-chlorotheophylline) and exhibits an unpleasant taste.

Taste masking is usually rather easy to achieve with conventional tablets, which may be coated with a suitable polymeric coating, and also in case of capsule formulations where the outer capsule shell itself provides a barrier which prevents contact between the active ingredient and the oral mucosa of the patient during administration. However, it is more challenging to mask the taste of a compound like dimenhydrinate when formulated as a dispersible, effervescent, or orally disintegrating dosage form or as granules for direct oral administration (“direct-to-mouth granules”), because in these cases the dosage unit is not swallowed as a whole, but the formulation comes into substantial contact with the oral mucosa.

In case of effervescent formulations, the drug typically dissolves in a larger amount of water, such as 200 mL, and in this diluted form, sufficient taste masking may sometimes, though not always, be achieved already by incorporating sweetening agents and flavours. Most challenging in terms of taste, though, is the formulation of the active ingredient in orally disintegrating tablets or granules for direct oral administration, since these dosage forms allow drug contact with the oral mucosa in concentrated form. These dosage form designs are highly desirable, though, because of their excellent swallowability, even without adding water.

Some compositions comprising poor-tasting drugs and taste-masking components and using alternative, more advanced taste-masking approaches are also known. For example, EP 0 839 528 discloses N-acetylcysteine tablet or granulate compositions formulated with cyclodextrins (complexing agents with cavities capable of hosting small molecules). In addition, the formulations comprise sweetening agents such as sorbitol and aspartame, and various flavours. However, cyclodextrins are expensive excipients, and for effective taste-masking require incorporation in relatively large amounts. Also, the use of sweeteners and flavouring agents to divert the patient from an active ingredient's unpleasant sensory attributes often requires the use of large amounts of these excipients to achieve a good taste-masking effect.

A generally more effective taste-masking approach is to provide a coating on the surface of the active ingredient. The coating serves as a physical barrier layer between the active ingredient and the patient's taste buds and olfactory receptors. In addition, a coating may be useful also to protect a sensitive or labile active ingredient during storage.

In principle, taste-masking coating may be polymeric film coatings or lipidic coatings. Polymeric coating systems are sprayed onto drug cores as aqueous or organic solutions or dispersions. A disadvantage of organic solvents is their need for special equipment and their negative impact on the environment. Aqueous coating systems also consume substantial energy, as the polymeric coating material must be heated above its film-forming temperature in order to coalesce, and the removal of water require more extensive drying than that of typical organic solvents. Moreover, many polymeric coating systems show curing effects, i.e. their properties change over time, so that the drug dissolution behaviour may become compromised during storage.

One example for an alternative composition comprising a poor-tasting drug and a polymer-containing coating that neither requires organic solvent nor aqueous dispersions can be found in EP 0 841 062 which discloses a granular preparation comprising melt-granules prepared by adhering a powdered medicine and optionally a powdered diluent (both ≤50 μm) to mononuclear core particles of a powdered low-melting oily substance (100-850 μm), preferably glycerolmonostearate (GMS) in a heated fluidized bed, using the resulting ‘stickiness’ of the oily substance's surface upon melting and surface-fusion. The resulting melt-granules are then coated in a similar manner with a finely powdered coating material, comprising a hydrophobic and oil-absorbing polymeric compound (<10 μm), preferably ethyl cellulose (EC), and a diluent such as talc (50 μm), which is also attached to the granules' surface by melting and surface-fusion. This preparation method is proposed as a means to avoid organic solvents or the risks that a water-based polymer-dispersion may pose to drugs (e.g. drugs prone to hydrolysis), as well as a means to overcome the ‘caking’ issue observed for GMS-melt-granulated drug particles when surface coated with talc only, instead of with talc plus EC (10 μm). This process does not yield, however, a polymeric coating in the stricter sense as it will not form a closed, or fused, polymeric topcoat. Instead, it is rather a ‘dusting’ or ‘powdering’ of the otherwise sticky and prone-to-caking lipid surface of the GMS-containing cores. And while EP 0 841 062 describes a specific melt-granulation method, the document is silent on the use of triglycerides and/or surfactants, and on the use of molten lipids as a coating material rather than talc plus polymers. Furthermore, the preparation method described therein requires careful selection and/or preparation of particles with specific particle size requirements (e.g. by grinding and sieving).

Lipidic coating systems, such as coatings based on waxes like carnauba wax or lipids such as stearic acid, do not require a solvent to be applied to drug-containing cores. They may often be used as melts in hot-melt coating processes. On the other hand, these types of coatings, due to the poor water solubility of its main constituents, also tend to have substantial negative impact on the drug's release profile, especially if rapid drug release is required. In such cases, wax and/or lipid coatings are often not successful.

Moreover, the stability of a lipidic or waxy taste-masking coating itself over time can also impact the release profile of the active ingredient. The conversion of an initially formed polymorph of a coating excipient to a thermodynamically more stable crystal form over time during the course of storage, sometimes also triggered by an exposure to different environmental conditions, can lead to significant and undesirable variations in the drug dissolution profile of the composition.

Furthermore, also the hot-melt processing conditions may be critical to temperature-sensitive drug compounds. Depending on the type of lipidic or waxy coating material, the coating process sometimes have to be conducted at temperatures of higher than 60° C., and sometimes also higher than 80° C., or even higher than 100° C. The same applies to EP 0 841 062. While describing a polymeric coating instead of a lipidic one, EP 0 841 062 works with the lipid glycerolmonostearate (GMS) as a ‘starter core’ material for their melt-granules, and employs rather high processing temperatures such as 90° C. inlet air temperature in order to melt, or soften, the GMS (melting point of about 71-73° C.).

WO2014/167124 and WO2015/193485 describe taste-masked compositions in the form of a hotmelt-coated particle comprising a core with a poorly tasting drug, such as drug crystals of N-acetylcysteine (NAC) or cores with an agglomerated active ingredient, and a coating comprising a triglyceride which is solid at room temperature and a surfactant. The particles exhibit rapid drug release and stable release profiles. Furthermore, the methods described in WO2014/167124 and WO2015/193485 allow the coating of core particles at moderate temperatures, thereby preventing the degradation of the thermolabile active ingredient. These documents thus overcome part of the above-mentioned problems of lipidic coatings.

As mentioned above, the taste perception can often be aggravated for active ingredients, in particular active pharmaceutical ingredients (APIs) with small particle sizes, due to their larger surface-to-volume ratio. In addition, some APIs which have a small or very small particle size (e.g. a mean particle size, or a DSO value, of about 50 μm to 150 μm, or about 10 μm to 50 μm, respectively) are particularly susceptible to electrostatic interactions, resulting in dust, uncontrolled build-up, caking, bridging, or adhesion to solid surfaces, both during manufacturing processes and during administration as a flowable dry powder composition. They are, thus, known to be difficult to handle, especially when they shall be orally administered. These issues can, in part, be solved by agglomerating the active ingredient to cores of larger size (e.g. 200-1000 μm) prior to applying a taste-masking coating; for instance, using roller-compaction such as proposed in WO2015/193485.

Alternatively, the active ingredient may also be agglomerated by surface-fusion based processes such as described in the above-mentioned EP 0 841 062, or other melt-agglomeration processes. Desai et. al. provides an overview on melt granulation techniques, such as fluidised hot melt granulation or spray-congealing, and points out that lipophilic melting materials such as GMS or stearic acid result in sustained release systems, and proposes to prepare fast release melt granules by utilizing water-soluble polymers and surfactants, such as PEG and poloxamers (Desai U. S., Chaudhari P. D., Bhaysar D. B., Chavan R. P.; “Melt Granulation: An Alternative to Traditional Granulation Techniques”. Indian Drugs 50(03), March 2013: 5-13).

It is an object of the invention to provide an improved method for the taste-masking of drug substances, especially for drug substances that display particular formulation challenges, such as small or very small API-particles, highly water-soluble particles, or APIs with unsatisfactory flow properties. Moreover, it is an object to provide an improved taste-masked dosage form of agglomerated drug particles which exhibits rapid drug dissolution and a stable dissolution profile. A further object is to provide improved pharmaceutical compositions comprising taste-masked agglomerated drug particles with rapid drug dissolution. A yet further object is to provide taste-masked compositions which may be manufactured at moderate temperatures, as well as processes by which taste-masked compositions of small or very small compounds may be prepared. Moreover, it is an object to overcome one or more of the limitations or disadvantages associated with the prior art. Further objects will become clear on the basis of the description and the claims.

These and further objects are achieved by the subject-matter as defined in the independent claims below, with particular embodiments outlined in the dependent claims.

### SUMMARY OF THE INVENTION

According to a first aspect, the invention provides a coated particle comprising a core and a coating, wherein the core comprises an agglomerated active ingredient, wherein the coating comprises a triglyceride which is solid at room temperature and a surfactant, and wherein the active ingredient in the core is melt-agglomerated. The coated particle allows for immediate release, i.e. rapid dissolution of the active ingredient in an aqueous medium. The coated particle is useful as a component of a pharmaceutical composition for oral use.

In a second aspect, the invention provides a method for the preparation of coated particles comprising a core with a melt-agglomerated active ingredient and a coating with a triglyceride which is solid at room temperature and a surfactant. The method includes the steps of (a) providing an active ingredient, (b) optionally, mixing the active ingredient with an anticaking free flow agent, (c) agglomerating the active ingredient, or optionally the mixture of the active ingredient and the anticaking free flow agent, with a molten composition comprising a triglyceride which is solid at room temperature and a surfactant such as to form a core comprising a melt-agglomerated active ingredient, (d) optionally, allowing the core of step (c) to cool down and solidify, (e) coating the core of step c, or optionally step (d), with a molten composition comprising a triglyceride which is solid at room temperature and a surfactant. It is preferably carried out in a fluid-bed coater or in an air flow bed coater. The product temperature may be kept at about 30° C. to 65° C. during the agglomeration step (c) and at about 20° C. to 50° C. during the coating step (e).

In a further aspect, the invention also relates to a coated particle comprising a core and a coating, wherein the core comprises an agglomerated active ingredient, and wherein the coating comprises a triglyceride which is solid at room temperature and a surfactant, said particle being obtainable by a method comprising the steps of (a) providing an active ingredient, (b) optionally, mixing the active ingredient with an anticaking agent, (c) agglomerating the active ingredient, or optionally the mixture of the active ingredient and the anticaking agent, with a molten composition comprising a triglyceride which is solid at room temperature and a surfactant such as to form a core comprising a melt-agglomerated active ingredient, (d) optionally, allowing the core of step (c) to cool down and solidify, and (e) coating the core with a molten composition comprising a triglyceride which is solid at room temperature and a surfactant.

In a yet further aspect, the invention provides a pharmaceutical composition comprising immediate-release coated particles comprising a melt-agglomerated active ingredient core and a coating with a triglyceride which is solid at room temperature and a surfactant. The composition may optionally be formulated as a dry flowable granular composition, such as a dispersible granular composition, an effervescent granular composition, a direct-to-mouth granular composition, or as a tablet, such as a dispersible tablet, an effervescent tablet, or an orally disintegrating tablet.

### Definitions

The following terms, or expressions, as used herein should normally be interpreted as outlined in this section, unless explicitly defined otherwise by the description or unless the specific context clearly indicates or requires otherwise:

Terms such as “about”, “approximately”, “ca.”, “essentially”, or “substantially” are meant to compensate for the variability allowed for in the technical field concerned and inherent in the respective products (e.g. in the pharmaceutical industry and in pharmaceutical products), such as differences in content due to manufacturing variation and/or time-induced product degradation. The terms in connection with an attribute or value include the exact attribute or the precise value, as well as any attribute or value typically considered to fall within a normal range or variability accepted in the technical field concerned.

The term “comprise” is to be construed in an open and inclusive sense, as “including, but not limited to”. The expressions “substantially consisting of” or “essentially consisting of” mean that no further components are added other than those listed. Nevertheless, very small amounts of other materials may be present, such as impurities.

The terms “drug”, “active agent”, “therapeutic agent”, “active pharmaceutical ingredient” (API), or “active ingredient” are used synonymously and refer to a compound or combination of compounds which are pharmaceutically active (or, as the case may be, nutraceutically active) against an undesired condition; or, in other words, ingredients which are administered, e.g. orally ingested, so as to cause an intentional therapeutic or prophylactic effect (e.g. an intentional change in a subject's physiology or psychology). It should be understood, though, that while certain triglycerides may theoretically also be administered as active ingredients in some cases, triglycerides, as used and described herein, are to be understood as an excipient, not as an active ingredient. The same applies to the surfactants as described herein.

The terms “a”, “an” or “the” do not exclude a plurality; i.e. these singular forms should be understood such as to include plural referents unless the context clearly indicates or requires otherwise. In other words, all references to singular characteristics or limitations of the present disclosure shall include the corresponding plural characteristic or limitation, and vice versa, unless explicitly specified otherwise or clearly implied to the contrary by the context in which the reference is made. The terms “a”, “an” or “the” thus have the same meaning as “at least one” or as “one or more” unless defined otherwise.

The expressions “one embodiment”, “an embodiment”, “a specific embodiment” and the like mean that a particular feature, property or characteristic, or a particular group, or combination, of features, properties or characteristics, as referred to in combination with the respective expression, is present in at least one of the embodiments of the invention. The occurrence of these expressions in various places throughout this description do not necessarily refer to the same embodiment. Moreover, the particular features, properties or characteristics may be combined in any suitable manner in one or more embodiments.

Any solubility provisions such as “soluble” or “freely soluble” as used herein shall be understood as aqueous solubilities at a temperature of 15-25° C. and ranked according to pharmacopoeial standards (e.g. European Pharmacopoeia, 8th edition) unless where specified otherwise:

| | Approximate volume of | |
|---|---|---|
| Solubility | solvent per 1 g of solute | |
| Very soluble | <1 | mL |
| Freely soluble | 1-10 | mL |
| Soluble | 10-30 | mL |
| Sparingly soluble | 30-100 | mL |
| Slightly soluble | 100-1000 | mL |
| Very slightly soluble | 1000-10000 | mL |
| Practically insoluble | >10000 | mL |

Any flow property provisions such as “passable”, “poor” or “very poor” as used herein shall be understood as flow properties measured and ranked according to pharmacopoeial standards (e.g. European Pharmacopoeia, 6th edition, chapter 2.9.36) unless where specified otherwise:

| Flow property | Angle of repose |
|---|---|
| Excellent | 25°-30° |
| Good | 31°-35° |
| Fair (aid not needed) | 36°-40° |
| Passable (may hang up) | 41°-45° |
| Poor (must agitate, vibrate) | 46°-55° |
| Very poor | 56°-65° |
| Very, very poor | >66° |

The term “room temperature” shall be understood as ranging from 15° C. to 25° C. (i.e. 20±5° C.), as for instance defined by the European Pharmacopoeia (Ph. Eur.) or by the WHO guidance ‘Guidelines for the Storage of Essential Medicines and Other Health Commodities” (2003). Typically, and unless specifically provided otherwise, this term does not imply any information beyond the temperature, e.g. no information such as whether or not this temperature is the result of dedicated climate control measures, or just of ambient conditions. Other temperature provisions, such as ‘about 37° C.’, and any other temperature provisions implying a controlled environment (such as in an oven, or in a standardized release tester like a USP Dissolution Apparatus type 2 paddle apparatus), are usually understood to be narrower; for instance, depending on the technical parameters of the device used ‘about 37° C.’ means 37±1° C., or even 37±0.5° C.

As used herein, a “triglyceride” is an ester derived from glycerol and three fatty acids. A triglyceride may also be referred to as a triacylglyceride.

### DETAILED DESCRIPTION OF THE INVENTION

In a first aspect, the invention provides a coated particle comprising a core and a coating, wherein the core comprises an agglomerated active ingredient, wherein the coating comprises a triglyceride which is solid at room temperature and a surfactant, and wherein the active ingredient in the core is melt-agglomerated. The particles allow for immediate release, or rapid dissolution, of the active ingredient in the presence of an aqueous medium. The coated particle is useful as a component of a pharmaceutical composition for oral use.

In a specific embodiment, the coated particle consists of one core and one coating layer. This means that the above-mentioned triglyceride/surfactant coating is directly applied to the core comprising the melt-agglomerated active ingredient without any intermediate layer(s) between core and coating, and without any additional coating layers on top of the triglyceride/surfactant coating.

In a further specific embodiment, the core of the coated particle may comprise only a single active ingredient. In an alternative embodiment, the core of the coated particle may comprise a plurality of active ingredients; for instance, a combination of two, three or four active ingredients.

In some embodiments of the invention, the surfactant in the coating may be a non-ionic surfactant, such as a polysorbate. Polysorbate 65 and polysorbate 85 are some of the preferred surfactants, in particular in combination with a saturated triglyceride selected from tripalmitin and tristearin. The coating comprises from 70 to 90 wt.-% triglyceride and from 10 to 30 wt.-% polysorbate, or in other words, the weight ratio of the triglyceride to the surfactant may be in the range from 70:30 to 90:10, in particular, in case of tripalmitin or tristearin and polysorbate 65.

The coating may be free of other constituents, such that the coating essentially consists of triglyceride and a polysorbate surfactant. In fact, one of the preferred coating compositions essentially consists of about 70 wt.-% of tristearate and about 30 wt.-% of polysorbate 65. Another preferred coating composition essentially consists of about 85 wt.-% of tripalmitin and about 15 wt.-% of polysorbate 65. Applying one of these coating compositions, for example a coating composition comprising from 70 to 90 wt.-% triglyceride and from 10 to 30 wt.-% polysorbate to the core with the melt-agglomerated active ingredient as a hot-melt (i.e. as a homogenously mixed, molten liquid) is surprisingly effective in simultaneously achieving effective taste-masking as well as rapid drug release, without release profile changes during storage. This applies in particular when the active ingredient is selected from dimenhydrinate, diphenhydramine, butylscopolamine, metformin hydrochloride, caffeine, paracetamol, ibuprofen, or hydrochlorothiazide, or any of their salts, isomers, polymorphs, and hydrates.

Alternatively, the coating may comprise one or more further excipients, such as one or more pore-forming agents, fillers, dyes or colouring agents, stabilisers, antioxidants, sweeteners, flavours, swelling agents, and the like. Preferably, however, the triglyceride and the surfactant together represent at least about 50 wt.-% of the coating, and more preferably at least about 70 wt.-%, or at least about 80 wt.-%, 90 wt.-%, or 95 wt.-%, respectively. According to a further preferred option, any further excipients are only incorporated at a level in which they are dissolved in, or miscible with, the molten triglyceride when the coating composition is sprayed onto the core particle.

In some embodiments, the active ingredient, prior to agglomeration, exhibits passable, poor, or very poor flow properties as defined by an angle of repose in the range of 41° to 45°, or 46° to 55°, or 56° to 65°, respectively as defined in in chapter 2.9.36, referring to “Powder flow”, of the European Pharmacopoeia, (like e.g. Ph. Eur., 6th edition). In some embodiments of the invention, prior to agglomeration the active ingredient exhibits passable flow properties at best, as indicated by an angle of repose of more than 40° as measured according to the recommended procedure in chapter 2.9.36 of the European Pharmacopoeia (Ph. Eur.), or more than 42°; or more than 45°; or more that 50°; and/or wherein the active ingredient exhibits passable or poor flow properties as defined by an angle of repose from 40° to 55° prior to agglomeration. In case, the core comprises a plurality of active ingredients, at least one of these, optionally all of these, exhibit the above described flow properties as defined by the angle of repose.

These passable, poor, or very poor flow properties of active ingredients are disadvantageous and a formulation-development challenge in that, usually, they do not allow, or at least hinder, direct oral administration without adding water as these active ingredients cannot easily be poured from a primary packaging (e.g. a sachet or vial) into the mouth of the patient. In addition, they negatively impact the processability of these active ingredients during manufacturing; for instance, easily forming lumps and aggregates and/or adhering to manufacturing equipment.

These passable, poor and very poor flow properties of an angle of repose of 41° to 45°, or 46° to 55°, and 56° to 65°, respectively, can alternatively be defined by i) a compressibility index of 21% to 25%, 26% to 31%, or 32% to 37%, respectively; or ii) by a Hausner ratio of 1.26 to 1.34, 1.35 to 1.45, or 1.46 to 1.59, respectively. Both the compressibility index and the Hausner ratio are measured as recommended in chapter 2.9.36, referring to “Powder flow”, of the European Pharmacopeia (e.g. Ph. Eur. 6th edition). In some embodiments of the invention, prior to agglomeration, the active ingredient exhibits a Hausner ratio of more than 1.25 as measured according to the recommended procedure in chapter 2.9.36 of the European Pharmacopoeia (Ph. Eur.), or more than 1.35; or more than 1.45; or more than 1.59; and/or wherein the active ingredient exhibits a Hausner ratio from 1.25 to 1.60 prior to agglomeration. Again, in case, the core comprises a plurality of active ingredients, at least one of these, optionally all of these, exhibit the above described flow properties as defined by the compressibility index and/or the Hausner ratio.

In a specific embodiment, the invention provides a coated particle comprising a core and a coating, wherein the core comprises an agglomerated active ingredient, wherein the coating comprises a triglyceride which is solid at room temperature and a surfactant, wherein the active ingredient in the core is melt-agglomerated and wherein prior to agglomeration the active ingredient exhibits an angle of repose of more than 50° as measured according to the recommended procedure in Ph. Eur.-chapter 2.9.36; and/or a Hausner ratio of more than 1.45 as measured according to the recommended procedure in Ph. Eur.-chapter 2.9.36.

In some embodiments, the active ingredient, prior to agglomeration, exhibits small or very small particle sizes, e.g. with a DSO value of the particle size distribution of from 10 μm to not more than 150 μm, or as described in further detail in the next paragraphs; for instance, a DSO value in the range of 10 μm to 50 μm for very small particles, and in the range of >50 μm to 150 μm for small particles. These small active ingredients, or small grades of active ingredients, are usually provided as fine powders and are oftentimes susceptible to electrostatic interactions and adhesion to container walls and are, thus, known to be difficult to handle, especially when intending to employ them in hot-melt coating processes, where process control is typically more difficult in comparison to aqueous coating/granulation compositions since more material is applied in the same time period, thus requiring an even more fine-tuned and robust selection of process parameters such as spray pressure, temperature and spray rate. The inventors now found that when first melt-agglomerating these small-particle APIs (e.g. DSO ca. 10-150 μm) prior to applying a melt-coating on top, even these small-particle APIs can be handled successfully and reproducibly. In other words, the present inventive coated particles with the melt-agglomerated core (as well as the method of preparation of these coated particles according to the second aspect of the invention as detailed further below) were found to be particularly advantageous for active ingredients exhibiting small or very small particle sizes (e.g. D50 ca. 10-150 μm).

In a specific embodiment of the invention, prior to agglomeration, the active ingredient exhibits a particle size distribution with a D50 value from 10 μm to 150 or from 10 μm to 70 or from 10 μm to 50 μm; and/or wherein prior to agglomeration the active ingredient exhibits a particle size distribution with a D90 value of from 90 to 500 or from 90 μm to 350 or from 90 μm to 250 or from 90 μm to 120 In a yet further specific embodiment, prior to agglomeration, the active ingredient exhibits a particle size distribution with a DSO value from 15 μm to 50 μm and a D90 value from 90 μm to 250 or from 90 μm to 120 In a yet further specific embodiment, prior to agglomeration, the active ingredient exhibits a particle size distribution with a DSO value from 100 μm to 150 μm and a D90 value from 250 μm to 350 or from 400 μm to 500 Preferably, the particle size distribution values were determined using a Camsizer® XT device (Retsch Technology GmbH, Haan, Germany) as described below.

As used herein, all particle size provisions relate to values, such as particle width values, as measured by dynamic image analysis, such as according to ISO 13322-2. Unless mentioned otherwise, the particle size values provided herein (either measured or calculated/derived from measured values) were determined using a Camsizer® XT device (Retsch Technology GmbH, Haan, Germany) equipped with the X-Jet plug-in cartridge and its related software.

The Camsizer® set-up employs a dynamic imaging technique, rather than actual ‘physical’ sieving of the particles. Samples are dispersed by pressurised air, optionally with the help of a vibrator, and passed through a gap illuminated by two bright, pulsed LED light sources. The images of the dispersed particles (more specifically of their shadows, or projections) are then recorded by two digital cameras and analysed for size and shape in order to determine a variety of length and width descriptors for the particles, as required e.g. by ISO norm 13322-2 (on particle size analysis via dynamic imaging); e.g. the width of the particle, i.e. the shortest chord of the measured set of maximum chords of a particle's projection (Camsizer parameter X_{c min}, also called the minimum largest chord diameter); or the maximum Feret diameter (Camsizer parameter X_{Fe max}); or the aspect ratio AR (Camsizer parameter b/l₃).

The particle width is the preferred particle size parameter in the present invention since this parameter is most closely related to physical screening using sieving manoeuvres. A particle with a width smaller than a sieve aperture is able to pass the sieve even if the length of such particle is larger than the width. Thus, the terms ‘particle size’ and ‘sieve diameter’ are nearly the same in the context of the invention and may be used interchangeably herein.

Using the particle width values, mass fractions within specific particle size ranges may then be derived, or cumulative mass fractions of all particles smaller than a specific particle size x (i.e. the fraction(s) which would pass the ‘virtual sieve’ of diameter x), as well as the weighted arithmetic mean particle size, and the D10, DSO and D90 values (i.e. the particle size of the ‘virtual sieve’ which is passed by a mass fraction of 10%, 50% and 90% of the particles, respectively); similar to usual sieve analysis, however with a far higher precision. In other words, a particle size distribution with a given DSO or D90 value, for example, means that 50% or 90%, respectively, of the particles are considered to have the given particle size value.

The Camsizer® XT device is preferred for particle size determinations according to the invention since it allows for the precise and reproducible analysis of particle sizes of fine powders down to 1 micrometre; a precision not achievable with conventional sieve analysis on sieving towers. This, however, should not be misinterpreted in such a way as to exclude, or prohibit, the use of conventional sieves or sieving towers for preliminary sieve diameter determinations and/or for the classification of particles. Furthermore, this does not exclude, or prohibit, the use of laser diffraction or any other established methods for preliminary particle size determinations. While in such cases the particle sizes determined may differ from those obtained with the Camsizer® set-up, the skilled person will appreciate, that preliminary evaluation of the particle size distribution is nonetheless possible in most cases; in case of doubt, the results obtained with a Camsizer® prevail.

The inventors further found that the coated particles according to the first aspect of the invention (as well as the method for their preparation according to the second aspect of the invention as detailed further below) are specifically advantageous for active ingredients which exhibit both passable, poor, or very poor flow properties as defined above, and small or very small particle sizes as defined above (for instance, a DSO value in the range of 10 μm to 50 μm for very small particles, and in the range of more than 50 μm to 150 μm for small particles).

In a specific embodiment, prior to agglomeration, the active ingredient exhibits passable flow properties at best, as indicated by an angle of repose of more than 40° as measured according to the recommended procedure in Ph. Eur.-chapter 2.9.36, or more than 42°; or more than 45°; or more that 50°; and a particle size distribution with a D50 value from 15 μm to 150 μm, or from 15 μm to 70 μm, or from 15 μm to 50 μm; and/or a particle size distribution with a D90 value of from 90 μm to 500 μm, or from 90 μm to 350 μm, or from 90 μm to 250 μm, or from 90 μm to 120 μm.

In a more specific embodiment of the invention, prior to agglomeration, the active ingredient exhibits of an angle of repose more than 50° as measured according to the recommended procedure in Ph. Eur.-chapter 2.9.36, and a particle size distribution with a DSO value from 15 μm to 50 μm as determined using a Camsizer® XT device (Retsch Technology GmbH, Haan, Germany), and/or a particle size distribution with a D90 value of from 90 μm to 120 μm.

It was further found by the inventors that the coated particles according to the first aspect of the invention are also suitable and advantageous for water-soluble active ingredients; for instance, active ingredients with a solubility of at least 1 mg/mL in water at a temperature between 15° C. and 25° C., or in other words, active ingredients which are at least sparingly soluble (as defined by the European Pharmacopoeia). In one embodiment of the invention, the active ingredient exhibits an aqueous solubility of at least 3 mg/mL in water at a temperature between 15 and 25° C. In one embodiment of the invention, the active ingredient exhibits an aqueous solubility of at least 200 mg/mL in water at a temperature between 15 and 25° C. In one embodiment of the invention, the active ingredient exhibits a solubility from at least 3 mg/mL to 200 mg/mL in water at a temperature between 15° C. and 25° C.

The core is typically solid, optionally semi-solid, at room temperature and comprises an active ingredient, for instance, a pharmacologically active ingredient, which is melt-agglomerated. The melt-agglomerated active ingredient may be crystalline, non-crystalline or partially crystalline. The active ingredient particles are associated by melt-agglomeration in the form of agglomerates such as pellets, micro-pellets, granules, or micro-particles using a molten binder composition, or melt-granulating composition. The shape of the core comprising, or consisting of, the melt-agglomerated active ingredient, primarily depends on the nature, composition and manufacturing method, e.g. on the shape and size of the API-particles and/or the viscosity of the melt-granulating composition. As used herein, the terms “melt-agglomerated”, “melt-agglomerate” or “melt-agglomeration” includes materials prepared by melt-granulation (i.e. a ‘build-up’ granulation using a molten lipid as the granulation liquid), pelletisation, melt-spraying, melt-extrusion and other methods known to the skilled person to be suited for agglomerating active ingredient particles with a molten binder composition. In one of the preferred embodiments, the cores are melt-agglomerated, or melt-granulated, by spraying a molten binder (specifically a molten binder comprising, or consisting of, a triglyceride which is solid at room temperature and a surfactant) onto the particles of the pharmacologically active ingredient, e.g. in a fluidized bed. Typically, the melt-agglomerated material, or core material, is prepared at temperatures above about 30° C., or above about 40° C., to prevent that the melt-agglomerated material (i.e. the core) gets too soft later on, when stored at room temperature.

Typically, the core comprising, or consisting of, the melt-agglomerated active ingredient exhibits a particle size distribution, as determined with the Camsizer® XT (see above), with a DSO-value below about 800 μm, and preferably below about 500 μm. In one embodiment, the core exhibits a particle size distribution with a DSO-value in the range from about 80 μm to about 800 μm, or from about 80 μm to about 600 μm, or from about 80 μm to about 500 μm. In one of the preferred embodiments, the core exhibits a particle size distribution with a DSO-value from about 80 μm to 400 μm, or from about 80 μm to 250 μm. In another of the preferred embodiments, the core exhibits a particle size distribution with a DSO-value from about 200 μm to 500 μm.

The molten binder, or the molten binder composition or melt-granulating composition, can comprise any pharmaceutically acceptable excipient suitable for the preparation of a melt-agglomerated active ingredient known to the killed person. In one of the preferred embodiments, the molten composition used to agglomerate, or granulate, the active ingredient particles, comprises a triglyceride which is solid at room temperature and a surfactant. In other words, with this embodiment of the invention, the core comprising the active ingredient further comprises a triglyceride which is solid at room temperature and a surfactant. In a more specific embodiment, the core essentially consists of the active ingredient, a triglyceride which is solid at room temperature and a surfactant. In a yet more specific embodiment, where the active agent is mixed with an anticaking agent, the core essentially consists of the active ingredient, an anticaking agent, a triglyceride which is solid at room temperature and a surfactant.

The inventors surprisingly found that despite the hydrophobic nature of said molten composition comprising a triglyceride which is solid at room temperature and a surfactant as described herein, melt-agglomerating the active ingredient particles with it leads to melt-granules which release the active ingredient rapidly (at least 75% of the active ingredient is dissolved within 45 minutes in 900 mL of an aqueous medium at 37° C., stirred at 50 rpm). Moreover, this rapid dissolution, or rapid release, was also found even when these melt-granules of the active ingredient were subsequently coated with a lipidic top-coating, for instance, with a molten coating composition also comprising a triglyceride which is solid at room temperature and a surfactant and a coating level of from 20 to 70 wt.-% relative to the total weight of the coated particles, such as about 40 wt.-%.

In a further specific embodiment, the core is substantially free of excipients other than the molten binder composition or melt-granulating composition and the optional anticaking agent; in particular, the core may be free of excipients other than the triglyceride which is solid at room temperature, the surfactant and the optional anticaking agent. For instance, in one of the preferred embodiments, the core is substantially free of powdered diluents such as starches, talc or lactose. This allows for the incorporation of higher amounts of active ingredient into the cores.

Optionally, the core comprises at least about 10 wt.-% of active ingredient relative to the weight of the core. In further preferred embodiments, the core comprises at least about 30 wt.-% of active ingredient, or at least about 40 wt.-%, or at least about 50 wt.-%, or at least about 55 wt.-%, or at least about 60 wt.-%, or at least about 65 wt.-%, or at least about 70 wt.-%, or even at least about 75 wt.-% of active ingredient; all these percentages being understood relative to the weight of the core. In a specific embodiment, the core comprises from about 45 wt.-% to 80 wt.-%, or from 60 wt.-% to 70 wt.-% of active ingredient relative to the weight of the core.

In particular, the core may comprise melt-agglomerates of an active ingredient with undesirable organoleptic properties such as bitterness, burning, stinging, metallic, or astringent mouthfeel when administered orally and which requires taste-masking. The active ingredient may, for example, be selected from analgesics, non-steroidal anti-inflammatory drugs, proton inhibitors, cough suppressants, antihistamines, antidiabetics, diuretics, stimulants, sedatives, decongestants, antiemetics, phosphate-binding agents, spasmolytics and sympathomimetics.

In one embodiment, the active ingredient is selected from dimenhydrinate, diphenhydramine, butylscopolamine, metformin, caffeine, paracetamol, ibuprofen, or hydrochlorothiazide, or any of their salts, isomers, polymorphs, and hydrates; e.g. diphenhydramine hydrochloride, butylscopolamine bromide or metformin hydrochloride.

In a specific embodiment, the active ingredient is dimenhydrinate. According to this specific embodiment, the invention provides a coated particle comprising a core and a coating, wherein the core comprises melt-agglomerated dimenhydrinate, and wherein the coating comprises a triglyceride which is solid at room temperature and a surfactant. In a more specific embodiment, the core comprises from about 60 wt.-% to 70 wt.-% of dimenhydrinate relative to the weight of the core; for instance, 63 wt.-%, 65 wt.-%, 66 wt.-%, or 68 wt.-%.

In yet another embodiment, the active ingredient comprised in the core is diphenhydramine, e.g. in the form of diphenhydramine hydrochloride. Optionally the core comprises from about 60 wt.-% to 70 wt.-% of diphenhydramine (e.g. diphenhydramine hydrochloride) relative to the weight of the core; for instance, 63 wt.-%, 65 wt.-%, 66 wt.-%, or 68 wt.-%.

In yet another embodiment, the active ingredient comprised in the core is butylscopolamine, e.g. in the form of butylscopolamine bromide. Optionally the core comprises from about 60 wt.-% to 70 wt.-% of butylscopolamine (e.g. butylscopolamine bromide) relative to the weight of the core; for instance, 63 wt.-%, 65 wt.-%, 66 wt.-%, or 68 wt.-%.

In yet another embodiment, the active ingredient comprised in the core is metformin, e.g. in the form of metformin hydrochloride. Optionally the core comprises from about 70 wt.-% to 80 wt.-% of metformin (e.g. metformin hydrochloride) relative to the weight of the core; for instance, 73 wt.-%, 75 wt.-%, 76 wt.-%, or 78 wt.-%.

In yet another embodiment, the active ingredient comprised in the core is caffeine. Optionally the core comprises from about 60 wt.-% to 70 wt.-% of caffeine relative to the weight of the core; for instance, 63 wt.-%, 65 wt.-%, 66 wt.-%, or 68 wt.-%.

In yet another embodiment, the active ingredient comprised in the core is hydrochlorothiazide. Optionally the core comprises from about 45 wt.-% to 55 wt.-% of hydrochlorothiazide relative to the weight of the core for instance, 48 wt.-%, 50 wt.-%, 62 wt.-%, or 54 wt.-%.

Optionally, the core of the coated particle further comprises an anticaking agent, also referred to as free flow agent. This additive shall prevent formation of lumps, facilitate raw material transport and improve flowability of the active ingredient particles during melt-granulation; especially for active ingredients with small particle sizes (e.g. DSO of 10 μm to 150 μm) and/or poor or very poor flow properties (as defined above; e.g. an angle of repose of more than 50°). It is especially of advantage, when the active ingredient is e.g. crystalline or has a branched shape and/or tends to form lumps. In one embodiment, the anticaking agent comprised in the core of the coated particle is selected from silicon salts, silicates, carbonates or stearates. In a further embodiment, the anticaking agent within the core is silica (silicon dioxide), preferably fumed silica or precipitated silica, or magnesium stearate. In a yet further specific embodiment, the anticaking agent is a hydrophilic fumed silica exhibiting a specific surface area in the range of 50-500 m²/g, or in the range of 200-300 m²/g; or a hydrophobic fumed silica treated with dimethyldichlorosilane (DDS). Examples of commercially available fumed silica grades suitable for the present invention include Aerosil® 200, Aerosil® 300, Aerosil® 380, and Aerosil® R972.

In a more specific embodiment, the core comprises an anticaking agent at an amount of at least 1.0 wt.-% based on the weight of the active ingredient in the core, preferably at least 1.5 wt.-%, more preferably at least 2.0 wt.-%. In a further embodiment, the core comprises an anticaking agent at an amount of not more than 5.0 wt.-% based on the weight of the active ingredient in the core, preferably not more than 4.0 wt.-%, more preferably not more than 3.0 wt.-%. For instance, in a specific embodiment, the core comprises an anticaking agent at an amount of from 0.01 wt.-% to 5.0 wt.-%, or from 0.1 wt.-% to 4.0 wt.-%, or from 0.5 wt.-% to 3.0 wt.-%, or from 1.0 wt.-% to 2.5 wt.-%, such 1.0 wt.-%, or 1.5 wt.-% or 2.0 wt.-%.

In one embodiment of the invention, the active ingredient is selected from dimenhydrinate, diphenhydramine (e.g. diphenhydramine hydrochloride), butylscopolamine (e.g. butylscopolamine bromide), metformin (e.g. metformin hydrochloride), caffeine, paracetamol, ibuprofen, or hydrochlorothiazide, or any of their salts, isomers, polymorphs, and hydrates; and the anticaking agent is fumed silica or magnesium stearate. In a more specific embodiment, the active ingredient is dimenhydrinate and the anticaking agent is fumed silica.

In order to obtain a core comprising a melt-agglomerated active ingredient, the active ingredient, optionally the active ingredient and the anticaking agent, is melt-agglomerated with a composition comprising a triglyceride which is solid at room temperature and a surfactant, more specifically a composition comprising said solid triglyceride and surfactant, and optional further components, in a homogeneously molten form. In a more specific embodiment, the active ingredient, optionally the active ingredient and the anticaking agent, is melt-agglomerated with a composition essentially consisting of a triglyceride which is solid at room temperature and a surfactant, more specifically a composition consisting of said solid triglyceride and surfactant in a homogeneously molten form. In order to melt-agglomerate the active ingredient, optionally the active ingredient and the anticaking agent, with such a homogenous mixture of the solid triglyceride and the surfactant, these two components are preferably provided as a molten, or pre-molten, lipid composition which serves as the granulation liquid during the preparation of the core comprising the melt-agglomerated active ingredient.

It has been surprisingly found by the inventors that these molten compositions comprising certain surfactants in combination with the solid triglyceride, in particular non-ionic surfactants such as polysorbates, may be used for melt-agglomerating the active ingredient, or drug, at relatively low temperatures while leading to cores which do not undergo any major changes with respect to their drug release behaviour; specifically, the melt-granules formed by agglomerating the active ingredient particles with these molten compositions comprising certain surfactants in combination with the solid triglyceride, in particular non-ionic surfactants such as polysorbates, keep their immediate release behaviour during storage (i.e. at least 75% of the active ingredient is dissolved, or released, within 45 minutes in 900 mL of an aqueous medium at 37° C., stirred at 50 rpm). Thus, the invention allows both the agglomeration and coating of temperature-sensitive drugs while achieving a product, or intermediate product, with significantly improved physical stability, and beneficial release properties for active ingredients that require both taste-masking and rapid onset of the effect after oral administration.

In one embodiment of the invention, the solid triglyceride used in the composition for melt-agglomerating the active ingredient and the solid triglyceride in the coating are the same and have the same characteristics as described above for the solid triglyceride in the coating.

In one embodiment of the invention, the surfactant used in the composition for melt-agglomerating the active ingredient and the surfactant in the coating are the same and have the characteristics as described above for the surfactants in the coating. Preferably, the surfactant used in the composition for melt-agglomerating the active ingredient is a polysorbate, preferably polysorbate 65 or polysorbate 85.

In one embodiment of the invention, the composition used for melt-agglomerating the active ingredient comprises from 70 to 90 wt.-% triglyceride and from 10 to 30 wt.-% polysorbate. In a more specific embodiment, the composition used for melt-agglomerating the active ingredient essentially consists of 70 to 90 wt.-% triglyceride and 10 to 30 wt.-% polysorbate. In other words, the weight ratio of the triglyceride to the surfactant in the core may be in the range from 70:30 to 90:10, in particular in case of tripalmitin or tristearin and polysorbate 65.

In one embodiment of the invention, the core comprises from 10 wt.-% to 70 wt.-% of active ingredient relative to the total weight of the coated particle. More specifically, the core comprises at least 15 wt.-% of active ingredient relative to the total weight of the total weight of the coated particle; or at least 20 wt.-%; or at least 30 wt.-%; or at least 40 wt.-%. In a specific embodiment, the core comprises about 40 wt.-% of dimenhydrinate relative to the total weight of the coated particle. In another specific embodiment, the core comprises about 40 wt.-% of diphenhydramine (e.g. diphenhydramine hydrochloride) relative to the total weight of the coated particle. In yet other specific embodiments, the core comprises about 40 wt.-% of butylscopolamine (e.g. butylscopolamine bromide); or about 60 wt.-% of metformin (e.g. metformin hydrochloride); about 40 wt.-% caffeine; or about 25 wt.-% hydrochlorothiazide relative to the total weight of the coated particle.

In one embodiment of the invention, the core essentially consists of:
the active ingredient, the triglyceride which is solid at room temperature and the surfactant; or
the active ingredient, the anticaking agent, the triglyceride which is solid at room temperature and the surfactant; or
the active ingredient, an anticaking agent selected from fumed silica and magnesium stearate, a triglyceride selected from tripalmitin and tristearin, and a polysorbate; or
an active ingredient selected from dimenhydrinate, diphenhydramine, butylscopolamine, metformin, caffeine, paracetamol, ibuprofen, or hydrochlorothiazide, or any of their salts, isomers, polymorphs, and hydrates, fumed silica, a triglyceride selected from tripalmitin and tristearin, and a polysorbate; or
an active ingredient selected from dimenhydrinate, diphenhydramine, butylscopolamine, metformin, caffeine, paracetamol, ibuprofen, or hydrochlorothiazide, or any of their salts, isomers, polymorphs, and hydrates, fumed silica, a triglyceride selected from tripalmitin and tristearin, and a polysorbate selected from polysorbate 65 or 85; or
an active ingredient selected from dimenhydrinate, diphenhydramine hydrochloride, butylscopolamine bromide, metformin hydrochloride, caffeine, paracetamol, ibuprofen, or hydrochlorothiazide, fumed silica, a triglyceride selected from tripalmitin and tristearin, and a polysorbate; or
an active ingredient selected from dimenhydrinate, diphenhydramine hydrochloride, butylscopolamine bromide, metformin hydrochloride, caffeine, paracetamol, ibuprofen, or hydrochlorothiazide, fumed silica, a triglyceride selected from tripalmitin and tristearin, and a polysorbate selected from polysorbate 65 or 85; or
dimenhydrinate, fumed silica, a triglyceride selected from tripalmitin and tristearin, and polysorbate 65.

More specifically, the core can comprise, or consist of, from about 63 to 69 wt.-% dimenhydrinate, from 1 to 2 wt.-% fumed silica and from about 30 to 35 wt.-% molten composition, relative to the total weight of the core, wherein the molten composition comprises from about 70 to 90 wt.-% of a triglyceride selected from tripalmitin and tristearin and from about 10 to 30 wt.-% polysorbate (e.g. polysorbate 65), or wherein more specifically the molten composition comprises from about 80 to 90 wt.-% of a triglyceride selected from tripalmitin and tristearin and from about 10 to 20 wt.-% polysorbate (e.g. polysorbate 65).

The coating is understood as a layer of material substantially enclosing the core, or at least enclosing the core in such a way as to cover the majority of the core surface. As it is an important objective of the invention to provide effective taste-masking of the active ingredient, it is preferred that at least 80% of the surface, or at least 90% of the surface, or at least 95% of the surface, or substantially all of the surface of the core is covered by the coating. At the same time, it will be appreciated by a person skilled in pharmaceutics that a bulk material comprising multiple coated particles according to the first aspect of the invention may include a minor fraction of particles whose coatings may not completely cover the cores, even though a majority of these particles exhibit substantially complete coatings.

Agglomerated core particles often have uneven, irregular surfaces, which, in some cases, may also be porous. The efficient application of an effective layer of coating is therefore usually challenging. This has particular consequences for agglomerated core particles comprising active ingredients which require taste-masking. It has been found that a coating composition comprising a triglyceride which is solid at room temperature and a surfactant is especially amenable towards the coating of agglomerated core particles in terms of processing, as well as achieving good taste-masking and adequate, sufficiently fast drug release times as described herein. It has been found that such a coating composition, when applied as a melt, readily covers the surface of the agglomerates, specifically the melt-agglomerates, and is particular effective in achieving taste masking.

Wax or lipid-based coatings typically provide taste-masking but also delay release of the active ingredient. In contrast, it has been found that the coated particles of the invention are taste-masked, but at the same time advantageously able to provide immediately release of the active ingredient. The particles are thus particularly suited for use in immediate-release pharmaceutical compositions. As defined herein, immediate release, or rapid dissolution, means a dissolution profile in which at least 75% of the active ingredient is dissolved in 900 mL of an aqueous medium at 37° C. and within 45 minutes, as determined using a USP Dissolution Apparatus type 2 (paddle apparatus) at stirring speed of 50 rpm. Preferably, the aqueous medium used for the dissolution test is 0.1 N hydrochloric acid with a pH 1.0, e.g. to mimic release in the stomach upon oral administration. The term immediate release may be used synonymously with the terms fast release, rapid release, rapid dissolution, quick release, or the like, as opposed to modified, slow, extended, controlled, or sustained release.

In a particular embodiment of the invention, using the above described dissolution test, the coated particles have a dissolution profile in which at least 75% of the active ingredient is dissolved within 30 minutes, or at least 85% within 30 minutes, or at least 85% with 15 minutes, which is also dependent on the properties (e.g. intrinsic solubility) of the active ingredient.

In one of the particularly useful embodiments, the active ingredient in the core is melt-agglomerated with a composition comprising the same qualitative components as the coating. Preferably, the active ingredient in the core is melt-agglomerated with a composition that is identical to the composition of the coating; (i.e. both qualitative and quantitative composition being the same). This also simplifies the method for the preparation of the coated particles advantageously, e.g. in that it allows the melt-coating step to be performed subsequent to the melt-agglomeration step without the need to exchange or clean equipment parts such as nozzles or pipes.

Within the context of the invention, and particularly applicable for the triglyceride in the coating and/or in the core (used for melt-agglomerating the active ingredient), the expression ‘solid at room temperature’ means that the lower limit of the melting range of the triglyceride is higher than about 20° C. More preferably, the lower limit of the melting range of the triglyceride is higher than about 35° C. In other preferred embodiments, the melting range is from about 40° C. to about 85° C., or from about 45° C. to about 70° C. If more than one triglyceride is present in the coating, at least one of them, representing a large fraction of the total triglyceride content in the coating (e.g. at least 50 wt.-% or more), should have a melting range according to one of these preferences. It is understood that the melting ranges are—as usually—given for a normal atmospheric pressure, e.g. approximately 1013 mbar.

In particular, native triglycerides often comprise fatty acid residues with different chain lengths and degrees of saturation, i.e. they represent mixtures of various chemically different triglycerides. For the sake of achieving more reproducible properties, triglycerides are therefore sometimes purified or semi-synthetically manufactured. Such more defined grades of triglycerides are also preferred according to the invention, both for use in the coating and for use in the core.

According to one of the preferred options, the triglyceride in the coating and/or the core is a substantially pure triglyceride, having a chemical purity of at least about 90%, i.e. comprising only a small fraction of triglycerides with other fatty acid residues than the main fraction. In particular, the chemical purity of the triglyceride may be at least about 95%, or at least about 97%, respectively.

According to another one of the preferred options, the triglyceride in the coating or the core is substantially saturated. In particular, the iodine value, which is a commonly used parameter to describe the degree of unsaturation in triglycerides and which reflects the mass of iodine in grams that is consumed by 100 grams of a triglyceride, may be lower than about 10, or not higher than about 5, or not higher than about 2, or not higher than about 1, respectively.

According to a further preferred option, the fatty acid residues of the triglyceride in the coating and/or the core are substantially the same, i.e. at least about 80%, or at least about 90%, or even at least about 95% of the acyl chains have the same number of carbon atoms and degree of saturation. Particularly useful are saturated triglycerides having acyl residues of 10 to 30 carbon atoms. Also preferred are saturated triglycerides having acyl residues with a chain length of 14 to 22 carbon atoms. Especially preferred are coatings comprising a triglyceride which comprises at least one acyl chain having 16 to 18 carbon atoms; and/or cores comprising an active ingredient that was melt-agglomerated with this type of triglyceride.

Its three fatty acid chains may be identical, as in trimyristin (or glyceryl trimyristate, melting point (Mp) ca. 56 57° C.), tripalmitin (or glyceryl tripalmitate, Mp ca. 61-65° C.), tristearin (or glyceryl tristearate, Mp ca. 70-73° C.), triarachidin (or glyceryl triarachidate Mp ca. 76-80° C.), or tribehenin (or glyceryl tribehenate, Mp ca. 82-86° C.). Especially preferred are coatings comprising a triglyceride which is selected from tripalmitin and tristearin; and/or cores comprising an active ingredient that was melt-agglomerated with this type of triglyceride. Optionally, two or more of these triglycerides may be used in combination.

Tripalmitin and tristearin, like many other saturated triglycerides, exhibit polymorphism. These triglycerides have an amorphous form and various crystalline forms, i.e. an unstable α-modification, a metastable β′-modification and a thermodynamically stable β-modification. Tripalmitin (in its stable β-form) typically has a melting range—as determined by Differential Scanning calorimetry (DSC)—of 61-65° C., whereas the melting range of tristearin is about 70-73° C.

Apart from the triglyceride, the coating comprises a surfactant. It has been surprisingly found by the inventors that coating compositions comprising certain surfactants in combination with a solid triglyceride, in particular non-ionic surfactants such as polysorbates, may be applied as hot-melt coatings at relatively low temperatures while leading to coated particles which do not undergo any major changes with respect to their drug release behaviour. This works particularly well with the above-mentioned cores comprising melt-agglomerated active ingredient. Thus, the invention allows both the agglomeration and the coating of temperature-sensitive drugs while achieving a product with significantly improved physical stability.

In one of the preferred embodiments, the coating comprises no further lipid or wax components other than the triglyceride described above. In particular, the coating may be free of higher melting components which require increased processing temperatures, or which could lead to an obstruction of the spray nozzle, such as carnauba wax.

Within the context of the invention, and particularly applicable for the surfactant in the coating and/or in the core (used for melt-agglomerating the active ingredient), the surfactant is optionally a non-ionic surfactant. Examples of pharmaceutically acceptable non-ionic surfactants include, without limitation, polysorbates, mono- and diglycerides of fatty acids, propylene glycol esters, sucrose fatty acid esters, polyglycerol esters, sorbitan fatty acid esters, polyoxyethylene sorbitan fatty acid esters, polyoxyethylene fatty acid ethers and poloxamers. In particular, polysorbates, such as polysorbate 65, have been found very suitable in combination with tristearin and tripalmitin. Optionally, two or more surfactants may be used in combination.

The surfactant, in particular the non-ionic surfactant, according to one of the preferred options, has a hydrophilic-lipophilic balance (HLB) value in the mid-range, in particular from about 5 to about 15, as described by Griffin (Calculation of HLB Values of Non-Ionic Surfactants, Journal of the Society of Cosmetic Chemists 5 (4): 249-56, 1954). Also preferred is a non-ionic surfactant with an HLB value in the range from about 6 to about 14, or from about 7 to about 13, or from about 8 to about 12, respectively. For example, polysorbate 65 exhibits an HLB value of about 10.5, and polysorbate 85 has an HLB value of about 11.

Alternatively, the surfactant in the coating and/or in the core may also be an ionic surfactant, such as a phospholipid or sodium dodecyl sulfate. Optionally, two or more surfactants may be used in combination. Further optionally, two or more surfactants comprising at least one ionic surfactant and at least one non-ionic surfactant may be used in combination.

In order to achieve a pronounced stabilising effect on the triglyceride in the coating and/or in the core, it is recommended to incorporate the surfactant at a surfactant-to-triglyceride ratio of at least about 0.05. More preferably, the ratio is in the range from about 0.05 to about 0.5, such as about 0.1, 0.15, 0.2, 0.25, 0.3, 0.35, 0.4, or 0.45. The coating composition and/or the composition used for melt-agglomerating the active ingredient may comprise, or even essentially consist of, from about 50 to 95 wt.-% of triglyceride and from about 5 to 50 wt.-% surfactant, in particular from about 70 to 95 wt.-% of triglyceride and from about 5 to 30 wt.-% surfactant.

Preferably, the surfactant is dissolved in, or miscible with, the triglyceride in the molten state, i.e. the surfactant is not incorporated at level which results in the formation of an emulsion or suspension in the molten state in which the coating composition is applied to the core particle. In this way, there is little risk for the coating and/or the melt-agglomerating (or melt-granulating) composition comprising the surfactant and triglyceride to separate into two phases at any time during or after the coating and/or agglomeration process, and a reduced risk for the clogging of nozzles during the coating and/or agglomeration process.

The thickness of the coating is selected with an eye on the size and shape of the core. For example, if, after melt-agglomeration, core particles are still shaped as flakes or needles which are to be taste-masked, this may require a larger relative amount of coating composition to be applied than in case of substantially spherical core particles having the same surface area. It will be appreciated by the skilled person that different weight ratios of the coating to the core are required for different core sizes and/or shapes to obtain the same coating thickness. In one exemplary embodiment, the weight of the coating is from about 20 to 70 wt.-% relative to the total weight of the coated particle (equalling from about 25 to about 230 wt.-% relative to the total weight of the uncoated core), or from about 30 to 50 wt.-% (equalling from about 42 to about 100 wt.-% relative to the total weight of the uncoated core).

In one embodiment, the coated particle according to the first aspect of the invention exhibits a particle size distribution with a D50-value from about 100 μm to about 1000 μm, preferably from about 100 μm to about 800 μm, more preferably from about 200 μm to 600 μm.

In one embodiment of the invention, the coated particle comprises, or consists of, about 40 wt.-% dimenhydrinate, about 0.8 wt.-% fumed silica, about 51 wt.-% triglycerides selected from tripalmitin and tristearin, and about 8.2 wt.-% polysorbate (e.g. polysorbate 65), relative to the total weight of the coated particle. In a more specific embodiment, this coated particle has a core comprising, or consisting of, about 66 wt.-% dimenhydrinate, about 1 wt.-% fumed silica, about 28 wt.-% triglycerides selected from tripalmitin and tristearin, and about 5 wt.-% polysorbate (e.g. polysorbate 65), relative to the total weight of the core (i.e. the yet uncoated melt-agglomerated active ingredient). In one of the preferred embodiments, the core comprising the dimenhydrinate and the fumed silica is melt-agglomerated, or melt-granulated, with the same triglyceride/polysorbate composition as is forming the coating.

In a further aspect, the invention provides a pharmaceutical composition comprising the coated particle according to the first aspect of the invention as described above, or typically a plurality of these coated particles. The coated particles as disclosed herein are particularly suitable for being incorporated in a composition for oral administration, in particular in the form of a dry flowable granular composition, such as a dispersible granular composition, an effervescent granular composition, a direct-to-mouth granular composition (i.e. a composition that can be ingested comfortably without additional water or other liquids or comestibles, either added to the granules prior to ingestion or used during ingestion), or as a tablet, such as a dispersible tablet, an effervescent tablet, or an orally disintegrating tablet.

In a specific embodiment, the pharmaceutical composition essentially consists of the coated particle according to the first aspect of the invention, or typically a plurality of these coated particles.

Particularly useful embodiments are oral formulations of the pharmaceutical compositions which consist of multiple units, or which disintegrate in the mouth of the patient into multiple units, such as direct-to-mouth granules or orally disintegrating tablet, because for these types of formulations the taste-masking effect of the multiple units is crucial for patient acceptability.

As used herein, direct-to-mouth granules are oral compositions designed for direct oral administration without adding water. Direct-to-mouth granules may represent mixtures of various types of multiple units, which units may be agglomerated and/or non-agglomerated particles. Often, such direct-to-mouth compositions represent mixtures of sweetening agents, such as sugars or sugar alcohols, flavours, and drug, any of which may be agglomerated or granulated.

An orally disintegrating tablet may be defined as solid single-unit dosage forms that rapidly disintegrates in the mouth of the patient without chewing, typically within less than about one or two minutes. Orally disintegrating tablets are usually pressed with lower compression forces than conventional tablets to obtain a higher porosity. Alternatively, their porosity may be increased by a drying or sublimation step for those tablets which contain a high amount of moisture or a sublimatable excipient. With regard to their formulation, the optimised use of disintegrants, such as commonly used crosslinked polymers, low-substituted celluloses, or effervescent couples, further contribute to rapid disintegration. Popular is also the use of highly water-soluble excipients which allow the actual dissolution of major parts of the formulations in the saliva, and which give a smoother mouthfeel compared to other formulations that disintegrate rapidly but leave mostly insoluble residues behind.

In a final aspect of the invention, the invention relates to a coated particle, wherein the coated particle comprises a core and a coating, wherein the core comprises an agglomerated active ingredient, and wherein the coating comprises a triglyceride which is solid at room temperature and a surfactant, said particle is obtainable by a method comprising the steps of (a) providing an active ingredient, (b) optionally, mixing the active ingredient with an anticaking agent, (c) agglomerating the active ingredient, or optionally the mixture of the active ingredient and the anticaking agent, with a molten composition comprising a triglyceride which is solid at room temperature and a surfactant such as to form a core comprising a melt-agglomerated active ingredient, (d) optionally, allowing the core of step (c) to cool down and solidify, and (e) coating the core with a molten composition comprising a triglyceride which is solid at room temperature and a surfactant. Optionally, the method further comprises step (f) curing the coated particle of step (e) at a temperature between 45° C. and 60° C., or between 50° C. and 55° C. It is understood that these steps are performed consecutively from step (a) to step (e) or optionally step (f).

The method for the preparation of the coated particle described above is the second aspect of the invention. The method comprises two main steps: a first step of preparing a core comprising an active ingredient by melt-agglomeration, also called melt-granulating; and a second step of melt-coating this core so as to form the coated particle according to the first aspect of the invention. In the first main step, the active ingredient, optionally blended with an anticaking agent, are sprayed with, or otherwise provided with, a molten composition, preferably a composition comprising a hot-melt of a triglyceride and a surfactant. This step is advantageous when handling drug substances that display formulation challenges such as active ingredients with small or very small particle sizes (e.g. DSO ca. 10-150 μm), highly water-soluble particles, or active ingredients with unsatisfactory flow properties. The thus produced melt-agglomerate comprising the associated active ingredient, represents the core, and can have the form of pellets, micro-pellets, granules, or micro-particles. In the second step, a molten composition comprising a holt-melt of a triglyceride and a surfactant is sprayed onto, or otherwise provided to, the core of the first step. In specific embodiments of the method for manufacturing the coated particles, the product temperature in the melt-agglomerating step (first main step) is at least 10° C. higher than in the coating step (second main step). Using melt-processes, the use of an organic solvent and the associated negative environmental, health and safety hazards may be avoided. Furthermore, as indicated above, using a composition as describe herein (i.e. comprising a triglyceride that is solid at room temperature and a surfactant, in particular a non-ionic surfactant), the resulting melt-agglomerated and melt-coated particles were found to be effectively taste-masked and at the same time exhibit both fast and storage-stable dissolution profiles.

In one embodiment, the method for the preparation of the coated particle described above, comprises the steps of (a) providing an active ingredient, (b) optionally, mixing the active ingredient with an anticaking agent, (c) agglomerating the active ingredient, or optionally the mixture of the active ingredient and the anticaking agent, with a molten composition comprising a triglyceride which is solid at room temperature and a surfactant such as to form a core comprising a melt-agglomerated active ingredient, (d) optionally, allowing the core of step (c) to cool down and solidify, and (e) coating the core of step (c), or optionally step (d), with a molten composition comprising a triglyceride which is solid at room temperature and a surfactant. Optionally, the method further comprises step (f) curing the coated particle of step (e) at a temperature between 45° C. and 60° C., or between 50° C. and 55° C. It is understood that these steps are performed consecutively from step (a) to step (e), or optionally step (a) to step (f).

The method may be carried out in any suitable coating equipment known to the skilled person; for example, the method may be performed in a fluid-bed coater, or in an air flow bed coater, e.g. the Ventilus V100 (Innojet Herbert Huettlin, Steinen, Germany).

In one embodiment of the method for the preparation, the method provides an additional step prior to step a), said step comprising, or consisting of, the measurement of the angle of repose of the active ingredient according to the recommended procedure in chapter 2.9.36 of the European Pharmacopoeia (Ph.Eur.). Said angle of repose test may be performed either by the provider of the active ingredient (and its results optionally be displayed in a certificate of analysis), and/or it may be performed ‘in-house’, and typically prior to providing the active ingredient in step a) for the melt-agglomeration step. The outcome of the angle-of-repose test may also be used to decide whether or not the addition of an anticaking agent to the active ingredient is advisable. In a more specific embodiment, the active ingredient provided in step a), and tested according to the recommended procedure in chapter 2.9.36 of the Ph.Eur., exhibits passable flow properties at best, as indicated by an angle of repose of more than 40° as measured according to the recommended procedure in chapter 2.9.36 of the European Pharmacopoeia; or more than 42°; or more than 45°; or more than 50°; and/or the active ingredient exhibits passable or poor flow properties as defined by an angle of repose from 40° to 55° prior to agglomeration. Such active ingredients are known to be difficult to handle due their unsatisfactory flow properties.

In respect of steps (c) or (e), melt-agglomerating or melt-coating, respectively, the nature of the triglyceride should also be taken into account. In case of a lower melting triglyceride such as tripalmitin, the product temperature may be kept between about 20° C. and 35° C., whereas in case of a coating composition based on a higher melting triglyceride such as tristearin, the product temperature may be kept between about 20° C. and 50° C., in particular between about 35° C. and 50° C., such as between about 40° C. and 48° C. In a specific embodiment, the product temperature is kept between about 30° C. and 65° C., preferably between about 35° C. and 60° C. or between about 40° C. and 55° C., while performing step (c); and/or at least step (c) is performed in a fluid-bed coater or air flow bed coater, optionally steps (c) and (d).

Preferably, the product temperature is kept between about 20° C. and 50° C., preferably between about 25° C. and 45° C. or between about 30° C. and 40° C., while performing step (e); and/or at least step (e) is performed in a fluid-bed coater or air flow bed coater, optionally steps (e) and (f). In a specific embodiment, the product temperature under step (c) is higher than the product temperature under step (e), preferably at least 5° C. higher, or at least 10° C. higher, or at least 15° C. higher; e.g. 6° C. higher, or 8° C. higher, or 9° C. higher, or 11° C. higher, or 12° C. higher, or 14° C. higher.

In one embodiment, steps (c) and (e), and optionally steps (d) and/or (f), are performed in the same device, such as in the same fluid-bed coater or air flow bed coater. This is advantageous in that the melt-agglomerated product does not have to be removed from the device used for melt-granulating first and then transferred into another device for the melt-coating step; like this contamination hazards are reduced, and times for cleaning and drying devices and parts thereof are reduced as well.

In one embodiment, the method for the preparation of the coated particle can be performed using qualitatively the same components for the molten composition for step (c) and step (e); in other words, the molten composition used for melt-agglomeration, or melt-granulation, of the active ingredient may comprise, or consist of, the same triglyceride(s) and surfactant(s) as the molten composition used for the coating. This is a very cost-effective way of manufacturing the coated particles according the first aspect of the invention in that fewer different raw materials need to be purchased, rather than a multitude thereof. In a specific embodiment, the active ingredient in the core is melt-agglomerated under step (c) with a molten composition that is identical to the composition of the coating applied under step (e); (i.e. both qualitative and quantitative composition being the same). This specific embodiment renders the process even more cost- and time-effective and simple, e.g. in that it allows the melt-coating step to be performed subsequent to the melt-agglomeration step without the need to exchange or clean equipment parts such as nozzles or pipes. Optionally, there is no dedicated break or time-period in-between the melt-granulation step and the subsequent melt-coating step; the coating step can be initiated right after the melt-agglomeration step, and is typically characterised by a reduced spray-rate so as to stop further agglomeration and instead allow the application of a top-coat.

The molten compositions under steps (c) and/or (e) can be applied by a spray-coating process (typically in a fluidized bed of the sprayed product), or by methods that drip, or gradually pour, the molten composition into the particle bed and, for instance, spread by mechanical agitation such as in a rotating drum. Preferably, though, the molten compositions under steps (c) and/or (e) are applied by a spray-coating process. In a specific embodiment, the molten compositions under steps (c) and (e) are applied by a spray-coating process, and the spray rate under step (c) is higher than the spray rate under step (e); for instance, the spray rate in step (c) can be about 1.5 to 2.5 times higher. It is understood, that the spray rates in both step (c) and (e) depends on the nature of active ingredient and the molten composition, the weight of the product bed, as well as the coating equipment and the desired outcome (e.g. higher spray rates for agglomeration than for coating). Furthermore, the spray rates depend on the product quantity and is higher when bigger quantities are manufactured. In an exemplary embodiment, the product quantity is from about 50 kg to 100 kg, or the product volume is from about 50 L to 100 L, and the spray rate under step (c) or at least at the start of step (c) is between 200 and 500 g/min while the spray rate under step (e) is between 400 and 700 g/min.

In most embodiments, in a final preparation step the coated particle of steps (e) or (f), is allowed to cool down and solidify. Optionally, this final preparation step is performed in a fluid-bed coater or air flow bed coater, such as in the same fluid-bed coater or air flow bed coater as used for steps (c) and/or (e).

One of the particular advantages of the invention is that the preparation method according to the second aspect of the invention provides an effective means for the agglomeration and coating of active ingredients with passable, poor, or very poor flow properties (as defined above; e.g. an angle of repose of more than 40° or more than 50°), and/or small or very small particle sizes (e.g. D50 ca. 10-150 μm, or even ca. 10-50 μm). Such active ingredients are prone to electrostatic adhesion to the processing equipment, as well as to self-aggregation; i.e. the active ingredient forming loose lumps with itself, held together mainly by electrostatic forces and thus, typically, exhibiting low mechanical stability and not being easily processed. As a result, they cannot easily be coated, and pose challenges in particular for taste-masking, e.g. by coated ‘powder nests’, or lumps, breaking apart upon oral administration and revealing the poorly tasting drug directly to the taste buds.

Typically, the poor flowability and/or small particle sizes result in substantially reduced coating efficiency since the fine-powdered, unagglomerated active ingredient ‘dust’ can hinder the coating equipment and coating process. Furthermore, the disintegration of any lumps formed by self-aggregation of the active ingredient during the coating process can lead to unacceptable inconsistencies in active ingredient content of the coated particles.

It has been found that the preparation method according to the second aspect of the invention, as well as the resulting coated particle(s) according to the first aspect of the invention overcomes such issues. Not only does the melt-agglomeration of the active ingredient in a first main step of the method allow for the preparation of cores with consist, reproducible active ingredient content, but also efficient coating is achieved when these cores comprising the melt-agglomerated active ingredient are sprayed with a molten coating composition comprising a triglyceride which is solid at room temperature and a surfactant. Importantly, the resulting coated particles are found to be effectively taste-masked and at the same time have fast and storage-stable dissolution profiles.

Another advantage of the invention is that the coating composition allows processing at rather low temperatures, thus being particularly suitable for the processing of active ingredients, e.g. drug substances, which are sensitive to degradation at elevated temperatures. Generally, it is not very suitable to coat thermally labile active ingredients with hot-melt coating compositions requiring a high coating temperature, such as coating compositions based on carnauba wax or other waxes.

Technical feasibility provided, all embodiments, including all specific or preferred embodiments, as described above in connection with the coated particle of the first aspect of the invention also apply to the method for the preparation of said coated particle according to the second aspect of the invention.

Further embodiments, options, and/or preferences are illustrated by the following examples.

### EXAMPLES

### Example 1: Hot-Melt Granulation of Dimenhydrinate Crystals

Pre-Mixing Dimenhydrinate with Aerosil® 200

Dimenhydrinate (DMH) with an angle of repose of 52° as measured according to Ph.Eur. chapter 2.9.36, and with a particle size distribution with a D50 value of about 40 μm to 45 μm as measured with a Camziser XT device (Retsch Technology GmbH, Haan, Germany) and Aerosil® 200 (2 wt.-% relative to the DMH-weight) were mixed for about 5 minutes using the Drum Hoop Mixer JEL RRM (J. Engelsmann AG, Ludwigshafen, Germany) at batch sizes ranging between 20 kg and 30 kg (e.g. 26 kg), classified through a 0.8 mm sieve, and subsequently mixed for about a further 10 minutes.

Melt-Agglomerating DMH with a Molten Composition of Tripalmitin and Polysorbate

The homogenised, pre-sieved powder-blend was then introduced into an air-flow bed coater (Ventilus V100, Innojet Herbert Huettlin, Steinen, Germany) which is equipped with a 10 to 20 μm Nylon stretch filter and adjusted to a fluid-spray gap, or nozzle gap, of 0.25 mm and fluidized. A molten mixture of 86 wt.-% of tripalmitin (Dynasan® 116) and 14 wt.-% of polysorbate 65 (Tween® 65) was prepared under stirring at a temperature of about 80 to 100° C. Maintaining an inlet air temperature of about 35 to 45° C. (e.g. about 40° C.) and a product temperature of about 45 to 55° C., a first part of the molten mixture (e.g. 30 to 35 wt.-%, or one third, of the total molten mixture) was sprayed onto the fluidized powder-blend at an initial spray rate of about 300 g/min and an inlet air amount of about 500 m³/h. After ca. 10% of said first part of the molten mixture was added and an initial ‘wetting’ of the fluidized powder bed was achieved, the spray rate was then increased to about 600 g/min and the inlet air amount adapted to about 600 m³/h until the first part of the molten mixture was added to the fluidized powder bed completely.

### Example 2: Hot-Melt Coating of the Melt-Granulated Dimenhydrinate Core Particles

After adjusting the inlet air temperature of the Ventilus V100 to about 20° C. to 30° C., the remainder, or second part of about two thirds, of the same molten mixture (i.e. 86 wt.-% tripalmitin and 14 wt.-% polysorbate 65), still stirred at a temperature of about 80 to 100° C., was sprayed onto the melt-granulated DMH core particles of example 1 at a spray rate ranging between 100 to 300 g/min (e.g. about 200 g/min), an inlet air amount of about 600 m³/h, and a product temperature of about 30° C. to 40° C. The process was stopped after the total amount of the molten mixture was sprayed (e.g. between 30 kg to 50 kg, such as 38 to 40 kg).

In total, the resulting coated particles with the melt-agglomerated DMH-cores (or briefly the coated DMH-particles) contained about 40 wt.-% DMH and about 60 wt.-% of the molten tripalmitin-polysorbate blend, and exhibited a particle size distribution with a DSO value of about 200 μm to 220 μm. It was confirmed by visual inspection that the melt-granulated DMH core granules were not agglomerated further (e.g. into double, triples, larger agglomerates) but merely top-coated with the second part of the molten tripalmitin-polysorbate blend.

Subsequently, the coated DMH particles were tested with respect to their taste and dissolution behaviour. The dissolution test was carried out in a USP Dissolution Apparatus type 2 (paddle apparatus). About 125 mg of the coated DMH particles were placed in dissolution vessels filled with 900 mL of 0.1 N hydrochloric acid (pH 1.0), and stirred at 50 rpm at 37±1° C. The DMH-content in the samples was analysed over time via HPLC with DAD (diode array detector).

| Sample | Drug Dissolution |
|---|---|
| | 54% within 1 min |
| | 97% within 5 min |
| | 98% within 10 min |
| | 99% within 15 min |
| | 99% within 30 min |

Taste-masking was evaluated by a panel of experts using a subjective organoleptic taste test. A small amount of water (1 mL) was injected into the oral cavity of a participant before intake of a sample. An amount of coated particles equivalent to a 50 mg dose of dimenhydrinate was used for taste testing. The time prior to the participant's sensation of a bitter or unpleasant taste was recorded.

In result, it was found that the coated DMH particles released not more than 60% of the active ingredient within 1 minute, but also very rapidly released more than 85% within 15 minutes and 99% within 30 minutes, respectively. The taste was found to be acceptable, as no bitter or unpleasant taste and/or anaesthetic effect could be detected within 60 s, i.e. the coating provided effective taste masking.

### Item List

1. A coated particle comprising a core and a coating, wherein the core comprises an agglomerated active ingredient, wherein the coating comprises a triglyceride which is solid at room temperature and a surfactant, and wherein the active ingredient in the core is melt-agglomerated.
2. The particle of item 1, wherein prior to agglomeration the active ingredient exhibits passable flow properties at best as indicated by an angle of repose of more than 40° as measured according to the recommended procedure in chapter 2.9.36 of the European Pharmacopoeia; or more than 42°; or more than 45°; or more than 50°; and/or wherein the active ingredient exhibits passable or poor flow properties as defined by an angle of repose from 40° to 55° prior to agglomeration.
3. The particle of any one of the preceding items, wherein prior to agglomeration the active ingredient exhibits a particle size distribution with a D50-value from 10 μm to 150 μm, or from 10 μm to 70 μm, or from 10 μm to 50 μm; and/or wherein prior to agglomeration the active ingredient exhibits a particle size distribution with a D90-value of from 90 μm to 500 μm, or from 90 μm to 350 μm or from 90 μm to 250 μm, or from 90 μm to 120 μm.
4. The particle of any one of the preceding items, wherein the active ingredient exhibits an aqueous solubility of at least 3 mg/mL in water at a temperature between 15 and 25° C.
5. The particle of any one of the preceding items, wherein the core further comprises an anticaking agent, preferably fumed silica or magnesium stearate.
6. The particle of any item 5, wherein the core comprises anticaking agent at an amount of at least 1.0 wt.-% based on the weight of the active ingredient in the core, preferably at least 1.5 wt.-%, more preferably at least 2.0 wt.-%.
7. The particle of any item 5, wherein the core comprises anticaking agent at an amount of not more than 10.0 wt.-% based on the weight of the active ingredient in the core, preferably not more than 5.0 wt.-%, more preferably not more than 3.0 wt.-%.
8. The particle of any item 5, wherein the core comprises anticaking agent at an amount of from 0.01 wt.-% to 10.0 wt.-%, or from 0.1 wt.-% to 5.0 wt.-%, or from 0.5 wt.-% to 3.0 wt.-%, or from 1.0 wt.-% to 3.0 wt.-%, such 1.0 wt.-%, or 1.5 wt.-% or 2 wt.-%.
9. The particle of any one of the preceding items, wherein the core comprising the active ingredient further comprises a triglyceride which is solid at room temperature and a surfactant.
10. The particle of any one of the preceding items, wherein the active ingredient, optionally the active ingredient and the anticaking agent, is melt-agglomerated with a composition comprising a triglyceride which is solid at room temperature and a surfactant.
11. The particle of item 10, wherein the active ingredient, optionally the active ingredient and the anticaking agent, is melt-agglomerated with a composition essentially consisting of a triglyceride which is solid at room temperature and a surfactant.
12. The particle of any one of items 10 or 11, wherein the triglyceride used for melt-agglomerating the active ingredient comprises at least one acyl chain having 16 to 18 carbon atoms and is preferably selected from tripalmitin and tristearin.
13. The particle of any one of items 10 to 12, wherein the surfactant used for melt-agglomerating the active ingredient is a polysorbate, preferably polysorbate 65 or polysorbate 85.
14. The particle of any one of the preceding items, wherein the composition used for melt-agglomerating the active ingredient comprises from 70 to 90 wt.-% triglyceride and from 10 to 30 wt.-% polysorbate.
15. The particle of any one of the preceding items, wherein the composition used for melt-agglomerating the active ingredient essentially consists of 70 to 90 wt.-% triglyceride and 10 to 30 wt.-% polysorbate.
16. The particle of any one of the preceding items, wherein the coated particle exhibits a particle size distribution with a D50-value from about 100 μm to about 1000 μm, preferably from about 100 μm to about 800 μm, more preferably from about 200 μm to 600 μm.
17. The particle of any one of the preceding items, wherein the active ingredient is selected from dimenhydrinate, diphenhydramine, butylscopolamine, metformin, caffeine, paracetamol, ibuprofen, or hydrochlorothiazide, or any of their salts, isomers, polymorphs, and hydrates.
18. The particle of any one of the preceding items, wherein the active ingredient is selected from dimenhydrinate, diphenhydramine hydrochloride, butylscopolamine bromide, metformin hydrochloride caffeine, paracetamol, ibuprofen, or hydrochlorothiazide.
19. The particle of any one of the preceding items, wherein the active ingredient is selected from dimenhydrinate, diphenhydramine hydrochloride, butylscopolamine bromide, metformin hydrochloride caffeine, paracetamol, ibuprofen, or hydrochlorothiazide; and wherein the anticaking agent is fumed silica or magnesium stearate.
20. The particle of any one of the preceding items, wherein the core comprises from 10 to 70 wt.-% of active ingredient relative to the total weight of the coated particle.
21. The particle of any one of the preceding items, wherein the core comprises at least 15 wt.-% of active ingredient relative to the total weight of the total weight of the coated particle; or at least 20 wt.-%; or at least 30 wt.-%; or at least 40 wt.-%.
22. The particle of any one of the preceding items, wherein the core essentially consists of:
   the active ingredient, the triglyceride which is solid at room temperature and the surfactant; or
   the active ingredient, the anticaking agent, the triglyceride which is solid at room temperature and the surfactant; or
   the active ingredient, an anticaking agent selected from fumed silica and magnesium stearate, a triglyceride selected from tripalmitin and tristearin, and a polysorbate; or
   an active ingredient selected from dimenhydrinate, diphenhydramine, butylscopolamine, metformin, caffeine, paracetamol, ibuprofen, or hydrochlorothiazide, or any of their salts, isomers, polymorphs, and hydrates, fumed silica, a triglyceride selected from tripalmitin and tristearin, and a polysorbate; or
   an active ingredient selected from dimenhydrinate, diphenhydramine, butylscopolamine, metformin, caffeine, paracetamol, ibuprofen, or hydrochlorothiazide, or any of their salts, isomers, polymorphs, and hydrates, fumed silica, a triglyceride selected from tripalmitin and tristearin, and a polysorbate selected from polysorbate 65 or 85; or
   an active ingredient selected from dimenhydrinate, diphenhydramine hydrochloride, butylscopolamine bromide, metformin hydrochloride, caffeine, paracetamol, ibuprofen, or hydrochlorothiazide, fumed silica, a triglyceride selected from tripalmitin and tristearin, and a polysorbate; or
   an active ingredient selected from dimenhydrinate, diphenhydramine hydrochloride, butylscopolamine bromide, metformin hydrochloride, caffeine, paracetamol, ibuprofen, or hydrochlorothiazide, fumed silica, a triglyceride selected from tripalmitin and tristearin, and a polysorbate selected from polysorbate 65 or 85; or
   dimenhydrinate, fumed silica, a triglyceride selected from tripalmitin and tristearin, and polysorbate 65.
23. The particle of any one of the preceding items, wherein the triglyceride in the coating comprises at least one acyl chain having 16 to 18 carbon atoms and is preferably selected from tripalmitin and tristearin.
24. The particle of any one of the preceding items, wherein the surfactant in the coating is a polysorbate, preferably polysorbate 65 or polysorbate 85.
25. The particle of any one of the preceding items, wherein the coating comprises from 70 to 90 wt.-% triglyceride and from 10 to 30 wt.-% polysorbate.
26. The particle of item 25, wherein the coating essentially consists of the triglyceride and the polysorbate.
27. The particle of any one of the preceding items, wherein the active ingredient in the core is melt-agglomerated with a composition comprising the same qualitative components as the coating.
28. The particle of any one of the preceding items, wherein the active ingredient in the core is melt-agglomerated with a composition that is identical to the composition of the coating.
29. The particle of any one of the preceding items, wherein the weight of the coating is from 20 to 70 wt.-% relative to the total weight of the coated particle.
30. The particle of any one of the preceding items, wherein the particle exhibits immediate release, and wherein the immediate release is defined by a dissolution profile in which at least 75% of the active ingredient is dissolved in 45 minutes, as determined using a USP Dissolution Apparatus type 2 (paddle apparatus) in 900 mL of an aqueous medium at 37° C. and at a stirring speed of 50 rpm.
31. The particle of any one of the preceding items, wherein the core comprises an agglomerated active ingredient at an amount of at least 10 wt.-% relative to the weight of the core.
32. A pharmaceutical composition comprising the coated particle of any one of items 1 to 31, or a plurality of these coated particles.
33. The pharmaceutical composition of item 32, being formulated as a dispersible granular composition, an effervescent granular composition, a direct-to-mouth granular composition, or as a dispersible tablet, an effervescent tablet, or an orally disintegrating tablet.
34. A method for the preparation of the coated particle of any one of items 1 to 31, comprising the steps of
   (a) providing an active ingredient,
   (b) optionally, mixing the active ingredient with an anticaking agent,
   (c) agglomerating the active ingredient, or optionally the mixture of the active ingredient and the anticaking agent, with a molten composition comprising a triglyceride which is solid at room temperature and a surfactant such as to form a core comprising a melt-agglomerated active ingredient,
   (d) optionally, allowing the core of step (c) to cool down and solidify, and
   (e) coating the core of step (c), or optionally step (d), with a molten composition comprising a triglyceride which is solid at room temperature and a surfactant, and
   (f) optionally, curing the coated particle of step (e) at a temperature between 45° C. and 60° C., or between 50° C. and 55° C.
35. The method of item 34 further comprising step
   (f) curing the coated particle of step (e) at a temperature between 45° C. and 60° C., or between 50° C. and 55° C.
36. The method of items 34 or 35 wherein the product temperature is kept between about 30° C. and 65° C., preferably between about 35° C. and 60° C. or between about 40° C. and 55° C., while performing step (c); and/or wherein at least step (c) is performed in a fluid-bed coater, optionally steps (c) and (d).
37. The method of any one of items 34 to 36, wherein the product temperature is kept between about 20° C. and 50° C., preferably between about 25° C. and 45° C. or between about 30° C. and 40° C., while performing step (e); and/or wherein at least step (e) is performed in a fluid-bed coater, optionally steps (e) and (f).
38. The method of any one of items 34 to 37, wherein the product temperature under step (c) is higher than the product temperature under step (e).
39. The method of items 34 to 39, wherein the product temperature is kept between about 30° C. and 65° C. while performing step (c), and kept between about 20° C. and 50° C. while performing step (e); and wherein the product temperature under step (c) is higher than the product temperature under step (e); and/or wherein at least step (c), or at least step (e) is performed in a fluid-bed coater.
40. The method of any one of items 34 to 39, wherein steps (c) and (e), and optionally steps (d) and/or (f), are performed in the same device, such as in the same fluid-bed coater.
41. The method of any one of items 34 to 40, wherein the active ingredient in the core is melt-agglomerated under step (c) with a molten composition that is identical to the composition of the coating applied under step (e).
42. The method of any one of items 34 to 41, wherein the molten compositions under steps (c) and/or (e) are applied by a spray-coating process.
43. The method of item 42, wherein the molten compositions under steps (c) and (e) are applied by a spray-coating process, and wherein the spray-rate under step (c) is higher than the spray-rate under step (e).
44. The method of any one of items 34 to 43, wherein in a final preparation step the coated particle of steps (e) or (f), is allowed to cool down and solidify; wherein optionally this final preparation step is performed in a fluid-bed coater, such as in the same fluid-bed coater as used for steps (c) and/or (e).
45. A coated particle comprising a core and a coating, wherein the core comprises an agglomerated active ingredient, and wherein the coating comprises a triglyceride which is solid at room temperature and a surfactant, said particle being obtainable by a method comprising the steps of
   (a) providing an active ingredient,
   (b) optionally, mixing the active ingredient with an anticaking agent,
   (c) agglomerating the active ingredient, or optionally the mixture of the active ingredient and the anticaking agent, with a molten composition comprising a triglyceride which is solid at room temperature and a surfactant such as to form a core comprising a melt-agglomerated active ingredient,
   (d) optionally, allowing the core of step (c) to cool down and solidify, and
   (e) coating the core with a molten composition comprising a triglyceride which is solid at room temperature and a surfactant.

1. A coated particle comprising a core and a coating,
wherein the core comprises an agglomerated active ingredient,
wherein the coating comprises a triglyceride which is solid at room temperature and a surfactant, and
wherein the active ingredient in the core is melt-agglomerated.

2. The particle of claim 1, wherein prior to agglomeration the active ingredient exhibits passable flow properties at best as indicated by an angle of repose of more than 40° as measured according to the recommended procedure in chapter 2.9.36 of the European Pharmacopoeia; or more than 42°; or more than 45°; or more than 50°; and/or wherein the active ingredient exhibits passable or poor flow properties as defined by an angle of repose from 40° to 55° prior to agglomeration.

3. The particle of claim 1, wherein prior to agglomeration the active ingredient exhibits a particle size distribution with a D50-value from 10 μm to 150 μm, or from 10 μm to 70 μm, or from 10 μm to 50 μm; and/or wherein prior to agglomeration the active ingredient exhibits a particle size distribution with a D90-value of from 90 μm to 500 μm, or from 90 μm to 350 μm or from 90 μm to 250 μm, or from 90 μm to 120 μm.

4. The particle of claim 1, wherein the core further comprises an anticaking agent, preferably fumed silica or magnesium stearate.

5. The particle of claim 1, wherein the active ingredient, optionally the active ingredient and the anticaking agent, is melt-agglomerated with a composition comprising a triglyceride which is solid at room temperature and a surfactant, or wherein the active ingredient, optionally the active ingredient and the anticaking agent, is melt-agglomerated with a composition essentially consisting of a triglyceride which is solid at room temperature and a surfactant.

6. The particle of claim 1, wherein the coated particle exhibits a particle size distribution with a D50-value from 100 μm to 1000 μm, preferably from 100 μm to 800 μm, more preferably from 200 μm to 600 μm.

7. The particle of claim 1, wherein the active ingredient is selected from dimenhydrinate, diphenhydramine, butylscopolamine, metformin, caffeine, paracetamol, ibuprofen, or hydrochlorothiazide, or any of their salts, isomers, polymorphs, and hydrates.

8. The particle of claim 1, wherein the core comprises from 10 to 70 wt.-% of active ingredient relative to the total weight of the coated particle; or at least 15 wt.-% of active ingredient relative to the total weight of the total weight of the coated particle; or at least 20 wt.-%; or at least 30 wt.-%; or at least 40 wt.-%.

9. The particle of claim 1, wherein the core essentially consists of:
the active ingredient, the triglyceride which is solid at room temperature and the surfactant; or
the active ingredient, the anticaking agent, the triglyceride which is solid at room temperature and the surfactant; or
the active ingredient, an anticaking agent selected from fumed silica and magnesium stearate, a triglyceride selected from tripalmitin and tristearin, and a polysorbate; or
an active ingredient selected from dimenhydrinate, diphenhydramine, butylscopolamine, metformin, caffeine, paracetamol, ibuprofen, or hydrochlorothiazide, or any of their salts, isomers, polymorphs, and hydrates, fumed silica, a triglyceride selected from tripalmitin and tristearin, and a polysorbate, optionally a polysorbate selected from polysorbate 65 or 85; or
dimenhydrinate, fumed silica, a triglyceride selected from tripalmitin and tristearin, and polysorbate 65.

10. The particle of claim 1, wherein the active ingredient in the core is melt-agglomerated with a composition comprising the same qualitative components as the coating.

11. The particle of claim 1, wherein the particle exhibits immediate release, and
wherein the immediate release is defined by a dissolution profile in which at least 75% of the active ingredient is dissolved in 45 minutes, as determined using a USP Dissolution Apparatus type 2 (paddle apparatus) in 900 mL of an aqueous medium at 37° C. and at a stirring speed of 50 rpm.

12. A pharmaceutical composition comprising the coated particle of claim 1, or a plurality of these coated particles, optionally being formulated as a dispersible granular composition, an effervescent granular composition, a direct-to-mouth granular composition, or as a dispersible tablet, an effervescent tablet, or an orally disintegrating tablet.

13. A method for the preparation of the coated particle of claim, comprising the steps of
a) providing an active ingredient,
b) optionally, mixing the active ingredient with an anticaking agent,
c) agglomerating the active ingredient, or optionally the mixture of the active ingredient and the anticaking agent, with a molten composition comprising a triglyceride which is solid at room temperature and a surfactant such as to form a core comprising a melt-agglomerated active ingredient,
d) optionally, allowing the core of step (c) to cool down and solidify, and
e) coating the core of step (c), or optionally step (d), with a molten composition comprising a triglyceride which is solid at room temperature and a surfactant, and
f) optionally, curing the coated particle of step (e) at a temperature between 45° C. and 60° C., or between 50° C. and 55° C.

14. The method of claim 13, wherein the product temperature is kept between about 30° C. and 65° C. while performing step (c), and kept between about 20° C. and 50° C. while performing step (e); and wherein the product temperature under step (c) is higher than the product temperature under step (e); and/or
wherein at least step (c), or at least step (e) is performed in a fluid-bed coater.

15. A coated particle comprising a core and a coating, wherein the core comprises an agglomerated active ingredient, and wherein the coating comprises a triglyceride which is solid at room temperature and a surfactant, said particle being obtainable by a method comprising the steps of
a) providing an active ingredient,
b) optionally, mixing the active ingredient with an anticaking agent,
c) agglomerating the active ingredient, or optionally the mixture of the active ingredient and the anticaking agent, with a molten composition comprising a triglyceride which is solid at room temperature and a surfactant such as to form a core comprising a melt-agglomerated active ingredient,
d) optionally, allowing the core of step (c) to cool down and solidify, and
e) coating the core with a molten composition comprising a triglyceride which is solid at room temperature and a surfactant.
